(19) United States
(12) Patent Application Publication
(10) Pub. No.: US 2005/0265928 A1
(43) Pub. Date: Dec. 1, 2005

(54) Pulmonary malarial vaccine

(75) Inventors: David A. Edwards, Boston, MA (US); Jean Sung, Cambridge, MA (US); Brian Pulliam, Brookline, MA (US); Eric Wehrenberg-Klee, Palo Alto, CA (US); Evan Schwartz, Tamarac, FL (US); Philip Dreyfuss, Wynnewood, PA (US); Sandeep Kulkarni, San Diego, CA (US); Erez Lieberman, Cambridge, MA (US)

Correspondence Address: PATREA L. PABST;PABST PATENT GROUP LLP, 400 COLONY SQUARE, SUITE 1200, ATLANTA, GA, 30361, US

(73) Assignee: President and Fellows of Harvard College Massachusetts

(21) Appl. No.: 11/125,010

(22) Filed: May 9, 2005

Related U.S. Application Data
(60) Provisional application No. 60/569,211, filed on May 7, 2004.

(51) Int. Cl. A61K 39/015; A61L 9/04; A61K 9/14

(52) U.S. Cl. 424/46; 424/272.1

(57) ABSTRACT

Particulate compositions for delivery, preferably pulmonary, which provide sustained release of antigens, preferably DNA and/or peptide and/or protein antigens, have been developed. In the preferred embodiment, aggregate nanoparticles are in the aerodynamic range of 1-5 microns diameter and fly deep into the lungs. As the aggregate particles degrade in the body, MSP-1 and AMA-1 proteins are released into the blood stimulating a humoural immune response. The individual particles in the range of 0.1 micron are preferentially phagocytosed by APCs which express the proteins encoded by AMA-1 and MSP-1 plasmid DNA thereby initiating the cellular immune response that is necessary for a complete immunity.

### CROSS-REFERENCE TO RELATED APPLICATIONS

This application claims priority to U.S. Provisional Application Ser. No. 60/569,211 filed May 7, 2004.

### BACKGROUND OF THE INVENTION

This invention is generally in the field of a method and compositions for vaccinating against diseases such as malaria, where vaccination strategies to date have been less than successful and there is a need for cheap and easy to administer vaccines.

Diseases such as malaria and tuberculosis are primarily diseases of third world countries. For example, malaria is a major health concern in South America, Africa, and most of the southern portion of Asia. There are 2.4 billion people at risk and between 300 and 500 million new cases each year, with 1.1 million deaths annually, most children. Drugs such as chloroquine and Malarone are too expensive, hard to achieve patient compliance with, and many strains have developed drug resistance to them.

During the 1960s and 1970s, early clinical studies showed that experimental vaccination with weakened malaria parasites could effectively immunize patients against a subsequent malaria infection. Because vaccines based on live, inactivated or killed malaria parasites are not currently economically or technically feasible, much of the research on vaccines focuses on identifying specific components or antigens of the malaria parasite that can start a protective immune response. Scientists encounter difficult obstacles in attempting to develop malaria vaccines, in terms of parasite biology, human immune responses, and both preclinical and clinical evaluation. Although four different species of protozoan parasites cause human malaria, most vaccine efforts have been directed toward Falciparum malaria because of its severity.

Parasites of the same species but isolated from different geographic locations may be genetically and immunologically distinct, so vaccines that protect against one geographic isolate may not protect against another. In addition, malaria parasites have complex life cycles with multiple distinct developmental stages creating potentially thousands of different antigens that could serve as targets of an immune response. Finally, because protection appears to require both antibody-mediated and cell-mediated immune responses, identifying delivery systems and formulations that stimulate all the aspects of immune reactivity represents an enormous technical challenge.

A sporozoite vaccine would protect against the infectious form injected into a person by a mosquito. But if a single sporozoite were to escape the body's immune defenses, it could eventually lead to full-blown disease. A merozoite (blood-stage) vaccine, in addition to safeguarding against that possibility, could prevent or diminish symptoms in persons already infected. A gametocyte (sexual stage) vaccine does not protect the person being vaccinated, but instead interrupts the cycle of transmission by inhibiting the further development of gametocytes once they-along with antibodies produced in response to the vaccine-are ingested by the mosquito. Although a sporozoite vaccine could be useful for protecting tourists or other persons exposed only briefly, the vaccine best suited for malarious parts of the world may well be a “cocktail” combining antigens from several parasite forms, and perhaps also from two or more species.

A number of candidate vaccine antigens have been identified from different developmental stages of the parasite (see FIG. 1), and some have advanced to the point of preliminary clinical evaluation. Researchers have largely focused on candidate vaccine antigens that are expressed on the parasite surface and/or are involved in some critical aspect of parasite development or disease. For example, the circumsporozoite (CS) protein is the dominant surface antigen of the sporozoite stage, and is believed to interact with receptors on the hepatocyte (human liver cell) surface during the initial infection.

Several antigens have been identified that are involved in binding merozoites to the human red blood cell or in the cell-invasion process. One, a merozoite surface protein (MSP-1), repeatedly has been found to elicit protective immunity in rodent and monkey models of malaria. Inhibition of such crucial steps in parasite growth would form a good strategy for a vaccine. Other studies have identified a parasite-derived molecule (PfEMP1) on the surface of infected red blood cells that mediates their binding to endothelial cells and other red cells. The parasite, however, has developed ways to prevent the immune system from attacking the infected red cell by regularly changing the structure of such surface proteins—a process known as antigenic variation. Recent studies of the P. falciparum genome have revealed two major families of variant genes, known as “var” (including PfEMP1) and “rif,” in P. falciparum expressed at different times during the course of an infection. Better understanding of antigenic variation may help scientists identify new strategies to interfere with parasite development.

Researchers are also investigating the immune mechanisms involved in severe malaria disease. For example, recent studies indicate that binding of plasmodium-infected red cells to a molecule found on the surface of cells within the placenta contributes to the adverse outcomes associated with malaria during a woman's first pregnancy, and may provide the basis for developing a vaccine to prevent this aspect of pathology. A few vaccine candidates, mostly based on sporozoite antigens, have undergone clinical trials. A vaccine made up of a combination of CS antigen and hepatitis B surface antigen showed sufficient protective efficacy in a small clinical trial to justify further testing in an endemic area. Only one candidate vaccine, Spf66, based on antigens from both merozoite and sporozoite stages, has undergone extensive field trials. It showed efficacy in early clinical trials in South America, but results from subsequent trials in Africa and Southeast Asia were not as promising.

In 1997, NIAID, the World Health Organization as well as other organizations and individuals from around the world, launched the Multilateral Initiative on Malaria (MIM). The NIH Fogarty International Center currently coordinates this program. Through cooperation and collaboration, the participants in this initiative hope to improve and expand research on malaria in Africa. There is only one malarial vaccine currently in clinical trials, using an adjuvant developed by Glaxo Smith Kline and in cooperation with the World Health Organization and National Institutes of Health. This is a vaccine combining a proprietary adjuvant with a protein antigen referred to as FMP-1.

It is therefore an object of the present invention to provide an alternative vaccine for diseases such as malaria.

It is a further object of the present invention to provide a vaccine that does not require multiple doses, provides sustained immunity, and induces more complete (humoral as well as cellular) immunity.

### SUMMARY OF THE INVENTION

Particulate compositions for delivery, preferably pulmonary, which provide sustained release of antigens such as malarial antigens, preferably DNA and/or peptide and/or protein antigens, have been developed. In the preferred embodiment, aggregate nanoparticles are in the aerodynamic range of 1-5 microns diameter and fly deep into the lungs. As the aggregate particles degrade in the body, MSP-1 and AMA-1 proteins are released into the blood stimulating a humoural immune response. The individual particles in the range of 0.1 micron are preferentially phagocytosed by APCs which express the proteins encoded by AMA-1 and MSP-1 plasmid DNA thereby initiating the cellular immune response that is necessary for a complete immunity.

### BRIEF DESCRIPTION OF THE DRAWINGS

FIG. 1 is a schematic of the various targets in the multi-stage life cycle of malaria.

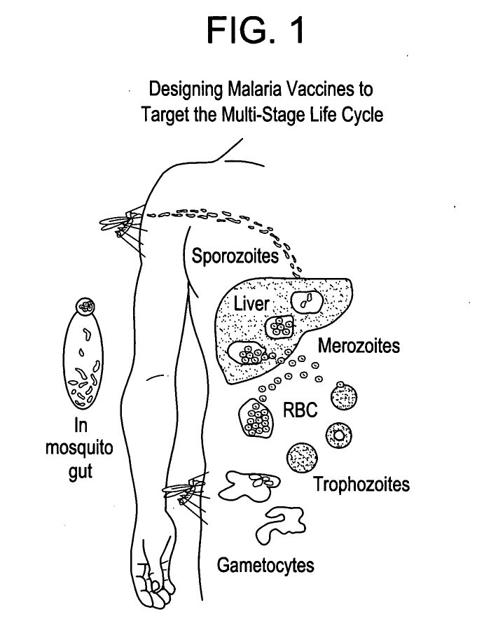

FIG. 2 is a schematic of the process for how sustained release of antigen from the surface of nanoparticles elicits humoral and cellular immunity.

### DETAILED DESCRIPTION OF THE INVENTION

I. Delivery Formulations

Particles

Particulate formulations for delivery of antigens, such as malarial antigens, have been developed. As published in PISCRBM, by Genentech in 1997, particle delivery substantially boosts protection. Particle size and charge both affect immunogenicity. For example, it is known that microparticles elicit an immune response and are easy to handle. Nanoparticles induce an improved cytotoxic T lymphocyte (“CTL”) responses.

Maximum response is obtained by binding of the antigen to the particle surfaces. Particles can also be made entirely of antigenic material or antigenic material can also be encapsulated within the particle. Nanoparticles are preferred, especially those which form structured aggregates. Numerous methods for making microparticles and nanoparticles, either of antigen (such as peptides, proteins, nucleic acids, small molecules) alone, antigen plus adjuvant, or antigen plus lipid, protein, amino acids, sugars or polymer, are available. In the preferred embodiment, nanoparticles of antigenic material (protein, peptide, nucleic acid and/or small molecules) are formulated into aggregates with a shell or matrix comprised of materials including polymers, lipids, sugars, amino acids and may also include antigenic material. Combinations of antigenic material can also be employed within the nanoparticles or microparticles.

Microparticles and Nanoparticles can be fabricated from different polymers (including proteins, polysaccharides, as well as biodegradable polymers such as polyhydroxy acids like poly(lactide-co-glycolide), polyhydroxyalkanoates, polyorthoesters, and polyanhydrides), non-biodegradable materials such as silica and polystyrene, lipids and/or the antigen to be delivered, using different methods.

a. Solvent Evaporation.

In this method the polymer is dissolved in a volatile organic solvent, such as methylene chloride. The antigenic agent (either soluble or dispersed as fine particles) is added to the solution, and the mixture is suspended in an aqueous solution that contains a surface active agent such as poly(vinyl alcohol). The resulting emulsion is stirred until most of the organic solvent evaporated, leaving solid microspheres. After stirring, the organic solvent evaporates from the polymer, and the resulting microspheres are washed with water and dried overnight in a lyophilizer. Microspheres with different sizes (1-1000 microns) and morphologies can be obtained by this method. This method is useful for relatively stable polymers like polyesters and polystyrene.

b. Hot Melt Microencapsulation.

In this method, the polymer is first melted and then mixed with the solid particles of drug that have been sieved to less than 50 microns. The mixture is suspended in a non-miscible solvent (like silicon oil), and, with continuous stirring, heated to 5° C. above the melting point of the polymer. Once the emulsion is stabilized, it is cooled until the polymer particles solidify. The resulting microspheres are washed by decantation with petroleum ether to give a free-flowing powder. Microspheres with sizes between one to 1000 microns are obtained with this method. The external surfaces of spheres prepared with this technique are usually smooth and dense. This procedure is used to prepare microspheres made of polyesters and polyanhydrides. However, this method is limited to polymers with molecular weights between 1000-50,000.

c. Solvent Removal.

This technique is primarily designed for polyanhydrides. In this method, the drug is dispersed or dissolved in a solution of the selected polymer in a volatile organic solvent like methylene chloride. This mixture is suspended by stirring in an organic oil (such as silicon oil) to form an emulsion. Unlike solvent evaporation, this method can be used to make microspheres from polymers with high melting points and different molecular weights. Microspheres that range between 1-300 microns can be obtained by this procedure. The external morphology of spheres produced with this technique is highly dependent on the type of polymer used.

d. Lipid Particles.

The particles bind a therapeutic, prophylactic or diagnostic agent, such as an antigen, in association with a charged lipid having a charge opposite to that of the agent. The charges are opposite upon association, prior to administration. In a preferred embodiment, the charges of the agent and lipid upon association, prior to administration, are those which the agent and lipid possess at pulmonary pH. The particle may have an overall net charge which can be modified by adjusting the pH of a solution of the agent, prior to association with the lipid. For example, at a pH of about 7.4 insulin has an overall net charge which is negative. Therefore, insulin and a positively charged lipid can be associated at this pH prior to administration to prepare a particle having an agent in association with a charged lipid wherein the charged lipid has a charge opposite to that of the agent. However, the charges on insulin can also be modified, when in solution, to possess an overall net charge which is positive by modifying the pH of the solution to be less than the pI of insulin (pI=5.5). As such, when insulin is in solution at a pH of about 4, for example, it will possess an overall net charge which is positive. The positively charged insulin can be associated with a negatively charged lipid, for example, 1,2-distearoyl-sn-glycero-3-[phosp-ho-rac-(1-glycerol)] (DSPG). Modification of the charge of the agent prior to association with the charged lipid, can be accomplished with many agents, particularly, proteins. For example, charges on proteins can be modulated by spray drying feed solutions below or above the isoelectric points (pI) of the protein. Charge modulation can also be accomplished for small molecules by spray drying feed solutions below or above the pKa of the molecule.

The particles can further comprise a carboxylic acid or carboxylic acid groups which are distinct from the agent and lipid. Carboxylic acids include the salts thereof as well as combinations of two or more carboxylic acids and/or salts thereof. In a preferred embodiment, the carboxylic acid is a hydrophilic carboxylic acid or salt thereof. Citric acid and citrates, such as, for example sodium citrate, are preferred. Combinations or mixtures of carboxylic acids and/or their salts also can be employed. Multivalent salts or their ionic components, such as a divalent salt, can be used. Examples include a salt of an alkaline-earth metal, such as, for example, calcium chloride. The particles of the invention can also include mixtures or combinations of salts and/or their ionic components. The particles can further comprise an amino acid. In a preferred embodiment the amino acid is hydrophobic.

The particles can be in the form of a dry powder suitable for inhalation. The particles can have a tap density of less than about 0.4 g/cm³, preferably less than about 0.1 g/cm³. Further, the particles can have a median geometric diameter of from about 5 micrometers to about 30 micrometers. In yet another embodiment, the particles have an aerodynamic diameter of from about 1 to about 5 micrometers.

The particles can be designed to possess a sustained release profile. This sustained released profile provides for prolonged residence of the administered bioactive agent in the lung and increases the amount of time in which therapeutic levels of the agent are present in the local environment or systemic circulation. “Sustained release”, as that term is used herein, refers to a release of active agent in which the period of release of an effective level of agent is longer than that seen with the same bioactive agent which is not associated with an oppositely charged lipid, prior to administration. In addition, a sustained release also refers to a reduction in the burst of agent typically seen in the first two hours following administration, and more preferably in the first hour, often referred to as the initial burst. In a preferred embodiment, the sustained release is characterized by both the period of release being longer in addition to a decreased burst. For example, a sustained release of insulin can be a release showing elevated levels out to at least 4 hours post administration, such as about 6 hours or more.

Agents which possess an overall net negative charge can be associated with a lipid which possesses an overall net positive charge. Agents which possess an overall net positive charge in association with a lipid which possesses an overall net negative charge, preferably in the pulmonary pH range, can be bound to a lipid such as 1,2-dipalmitoyl-sn-glycero-3- -[phospho-rac-(1-glycerol)] (DPPG) which possesses an overall net negative charge. “Pulmonary pH range”, as that term is used herein, refers to the pH range which can be encountered in the lung of a patient. Typically, in humans, this range of pH is from about 6.4 to about 7.0, such as from 6.4 to about 6.7 pH values of the airway lining fluid (ALF) have been reported in “Comparative Biology of the Normal Lung”, CRC Press, (1991) by R. A. Parent and range from 6.44 to 6.74)

“Charged lipid” as that term is used herein, refers to lipids which are capable of possessing an overall net charge. The charge on the lipid can be negative or positive. The lipid can be chosen to have a charge opposite to that of the active agent when the lipid and active agent are associated. In a preferred embodiment the charged lipid is a charged phospholipid. Preferably, the phospholipid is endogenous to the lung or can be metabolized upon administration to a lung endogenous phospholipid. Combinations of charged lipids can be used. The combination of charged lipid also has an overall net charge opposite to that of the bioactive agent upon association.

The charged phospholipid can be a negatively charged lipid such as, a 1,2-diacyl-sn-glycero-3-[phospho-rac-(1-glycerol)] and a 1,2-diacyl-sn-glycerol-3-phosphate. Specific examples of negatively charged phospholipids include, but are not limited to, 1,2-distearoyl-sn-glycero-3-[phospho-rac- -(1-glycerol)] (DSPG), 1,2-dimyristoyl-sn-glycero-3-[phospho-rac-(1-glycer-ol)] (DMPG), 1,2-dipalmitoyl-sn-glycero-3-[phospho-rac-(1-glycerol)] (DPPG), 1,2-dilauroyl-sn-glycero-3-[phospho-rac-(1-glycerol)] (DLPG), 1,2-dioleoyl-sn-glycero-3-[phospho-rac-(1-glycerol)] (DOPG), 1,2-dimyristoyl-sn-glycero-3-phosphate (DMPA), 1,2-dipalmitoyl-sn-glycero-3-phosphate (DPPA), 1,2-dioleoyl-sn-glycero-3-phosphate (DOPA), 1,2-distearoyl-sn-glycero-3-phosphate (DSPA), and 1,2-dilauroyl-sn-glycero-3-phosphate (DLPA).

The charged lipid can be a positively charged lipid such as a 1,2-diacyl-sn-glycero-3-alkylphosphocholine and a 1,2-diacyl-sn-glycero-3- -alkylphosphoalkanolamine. Specific examples of this type of positively charged phospholipid include, but are not limited to, 1,2-dipalmitoyl-sn-glycero-3-ethylphosphocholine (DPePC), 1,2-dimyristoyl-sn-glycero-3-ethylphosphocholine (DMePC), 1,2-distearoyl-sn-glycero-3-ethylphosphocholine (DSePC), 1,2-dilauroyl-sn-glycero-3-ethylphosphocholine (DLePC), 1,2-dioleoyl-sn-glycero-3-ethylphosphocholine (DOePC), 1,2-dipalmitoyl-sn-glycero-3-ethylethano-lamine (DPePE), 1,2-dimyristoyl-sn-glycero-3-ethylphosphoethanolamine (DMePE), 1,2-distearoyl-sn-glycero-3-ethylphosphoethanolamine (DSePE), 1,2-dilauroyl-sn-glycero-3-ethylphosphoethanolamine (DLePE), and 1,2-dioleoyl-sn-glycero-3-ethylphosphoethanolamine (DOePE). Other charged lipids suitable include those described in U.S. Pat. No. 5,466,841to Horrobin et al. issued on Nov. 14, 1995, U.S. Pat. Nos. 5,698,721 and 5,902,802 to Heath issued Dec. 16, 1997 and May 11, 1999, respectively, and U.S. Pat. No. 4,480,041 to Myles et al. issued Oct. 30, 1984, the entire contents of all of which are incorporated herein by reference.

The particles can be prepared by spray drying. For example, a spray drying mixture, also referred to herein as “feed solution” or “feed mixture”, which includes the bioactive agent and one or more charged lipids having a charge opposite to that of the active agent upon association are fed to a spray dryer. For example, when employing a protein active agent, the agent may be dissolved in a buffer system above or below the pI of the agent. Specifically, insulin for example may be dissolved in an aqueous buffer system (e.g., citrate, phosphate, acetate, etc.) or in 0.01 N HCl. The pH of the resultant solution then can be adjusted to a desired value using an appropriate base solution (e.g., 1 N NaOH). In one preferred embodiment, the pH may be adjusted to about pH 7.4. At this pH insulin molecules have a net negative charge (pI=5.5). In another embodiment, the pH may be adjusted to about pH 4.0. At this pH insulin molecules have a net positive charge (pI=5.5). Typically the cationic phospholipid is dissolved in an organic solvent or combination of solvents. The two solutions are then mixed together and the resulting mixture is spray dried.

For a small molecule active agent, the agent may be dissolved in a buffer system above or below the pKa of the ionizable group(s). For example, albuterol sulfate or estrone sulfate, for example, can be dissolved in an aqueous buffer system (e.g., citrate, phosphate, acetate, etc.) or in sterile water for irrigation. The pH of the resultant solution then can be adjusted to a desired value using an appropriate acid or base solution. If the pH is adjusted to about pH 3 to about pH 8 range, estrone sulfate will possess one negative charge per molecule and albuterol sulfate will possess one positive charge per molecule. Therefore, charge interaction can be engineered by the choice of an appropriate phospholipid. Typically the negatively charged or the positively charged phospholipid is dissolved in an organic solvent or combination of solvents and the two solutions are then mixed together and the resulting mixture is spray dried.

Suitable organic solvents that can be present in the mixture being spray dried include, but are not limited to, alcohols for example, ethanol, methanol, propanol, isopropanol, butanols, and others. Other organic solvents include, but are not limited to, perfluorocarbons, dichloromethane, chloroform, ether, ethyl acetate, methyl tert-butyl ether and others. Aqueous solvents that can be present in the feed mixture include water and buffered solutions. Both organic and aqueous solvents can be present in the spray-drying mixture fed to the spray dryer. In one embodiment, an ethanol water solvent is preferred with the ethanol:water ratio ranging from about 50:50 to about 90:10. The mixture can have a, acidic or alkaline pH. Optionally, a pH buffer can be included. Preferably, the pH can range from about 3 to about 10.

The total amount of solvent or solvents being employed in the mixture being spray dried generally is greater than 99 weight percent. The amount of solids (drug, charged lipid and other ingredients) present in the mixture being spray dried generally is less than about 1.0 weight percent. Preferably, the amount of solids in the mixture being spray dried ranges from about 0.05% to about 0.5% by weight. Using a mixture which includes an organic and an aqueous solvent in the spray drying process allows for the combination of hydrophilic and hydrophobic components, while not requiring the formation of liposomes or other structures or complexes to facilitate solubilization of the combination of such components within the particles.

Suitable spray-drying techniques are described, for example, by K. Masters in “Spray Drying Handbook”, John Wiley & Sons, New York, 1984. Generally, during spray-drying, heat from a hot gas such as heated air or nitrogen is used to evaporate the solvent from droplets formed by atomizing a continuous liquid feed. Other spray-drying techniques are well known to those skilled in the art. In a preferred embodiment, a two-fluid atomization technique is employed. In another embodiment, rotary atomization is used. An example of a suitable spray dryer using rotary atomization includes the Mobile Minor spray dryer, manufactured by Niro, Denmark. The hot gas can be, for example, air, nitrogen or argon. Another example of a suitable spray dryer using two-fluid atomization includes the SD-06 spray-dryer manufactured by LabPlant, UK.

Preferably, the particles are obtained by spray drying using an inlet temperature between about 90 degrees C. and about 400 degrees C. and an outlet temperature between about 40 degrees C. and about 130 degrees C. The spray dried particles can be fabricated with a rough surface texture to reduce particle agglomeration and improve flowability of the powder. The spray-dried particle can be fabricated with features which enhance aerosolization via dry powder inhaler devices, and lead to lower deposition in the mouth, throat and inhaler device.

e. Hydrogel Microspheres.

Microspheres made of gel-type polymers, such as alginate, are produced through traditional ionic gelation techniques. The polymers are first dissolved in an aqueous solution, mixed with barium sulfate or some bioactive agent, and then extruded through a microdroplet forming device, which in some instances employs a flow of nitrogen gas to break off the droplet. A slowly stirred (approximately 100-170 RPM) ionic hardening bath is positioned below the extruding device to catch the forming microdroplets. The microspheres are left to incubate in the bath for twenty to thirty minutes in order to allow sufficient time for gelation to occur. Microsphere particle size is controlled by using various size extruders or varying either the nitrogen gas or polymer solution flow rates. Chitosan microspheres can be prepared by dissolving the polymer in acidic solution and crosslinking it with tripolyphosphate. Carboxymethyl cellulose (CMC) microspheres can be prepared by dissolving the polymer in acid solution and precipitating the microsphere with lead ions. In the case of negatively charged polymers (e.g., alginate, CMC), positively charged ligands (e.g., polylysine, polyethyleneimine) of different molecular weights can be ionically attached.

The nanoparticles can contain from 0.01% (w/w) to about 100% (w/w) of antigenic material (dry weight of composition). The amount of protein, peptide, nucleic acid or small molecule material used will vary depending on the desired effect and release levels. Combinations of antigenic material can be employed.

Particles, preferably nanoparticles, can be assembled into structured aggregates on the micron size scale, with a shell or matrix consisting of a mixture of lipophilic and/or hydrophilic molecules (normally pharmaceutical “excipients”). The nanoparticles can be formed in the aforementioned methods and incorporate nucleic acid and/or peptide and/or protein and/or small molecule antigens as the body of the particle, on the surface of the particles or encapsulated within the particles. The aggregate particle shell or matrix can include pharmaceutical excipients such as lipids, amino acids, sugars, polymers and may also incorporate nucleic acid and/or peptide and/or protein and/or small molecule antigens. Combinations of antigenic material can also be employed. These aggregate particles can be formed in the following methods.

a. Porous Nanoparticle Aggregate Particles.

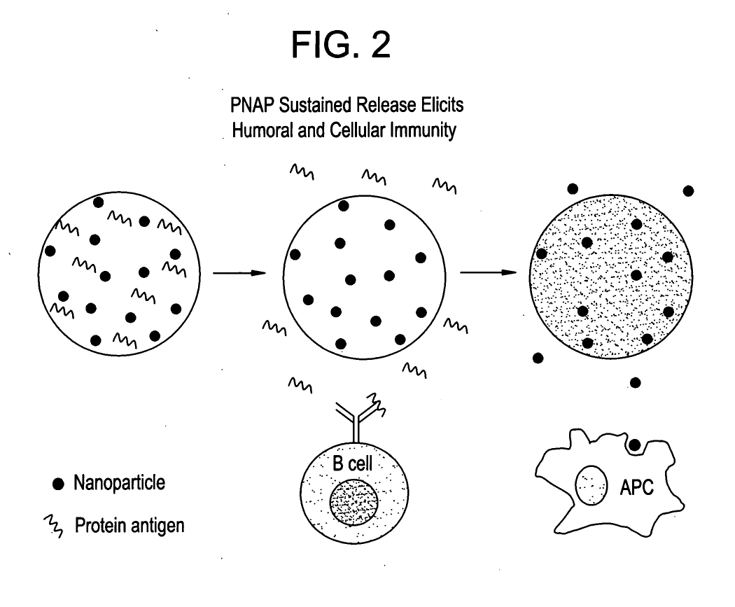

U.S. patent application Ser. No. 20040062718 describes a preferred method of making porous nanoparticle aggregate particles for use as vaccines. Antigen can be associated with the nanoparticles by making up the nanoparticles, being bound to the surface of the nanoparticles or encapsulated within the nanoparticles or it can be incorporated in the shell of the microparticles, as depicted in FIG. 2, which then elicits both humoral and cellular immunity.

These particles aggregate, as described by Edwards, et al., Proc. Natl. Acad. Sci. USA 19, 12001-12005 (2002), to produce larger particles of smaller subunit particles (called Trojan particles because they maintain the unique properties of their smaller subunits while also maintaining key characteristics of larger particles). The agent may be encapsulated within the subunit particles or within the larger particles made from the smaller particle aggregates.

The particles, also referred to herein as powder, can be in the form of a dry powder suitable for inhalation. In a particular embodiment, the particles can have a tap density of less than about 0.4 g/cm³. Particles which have a tap density of less than about 0.4 g/cm³ are referred to herein as “aerodynamically light particles.” More preferred are particles having a tap density less than about 0.1 g/cm³. Aerodynamically light particles have a preferred size, e.g., a volume median geometric diameter (VMGD) of at least about 5 microns. In one embodiment, the VMGD is from about 5 microns to about 30 microns. In another embodiment, the particles have a VMGD ranging from about 9 microns to about 30 microns. In other embodiments, the particles have a median diameter, mass median diameter (MMD), a mass median envelope diameter (MMED) or a mass median geometric diameter (MMGD) of at least 5 microns, for example from about 5 microns to about 30 microns. Aerodynamically light particles preferably have “mass median aerodynamic diameter” (MMAD), also referred to herein as “aerodynamic diameter”, between about 1 microns and about 5 microns. In one embodiment, the MMAD is between about 1 microns and about 3 microns. In another embodiment, the MMAD is between about 3 microns and about 5 microns.

In another embodiment, the particles have an envelope mass density, also referred to herein as “mass density” of less than about 0.4 g/cm³. The envelope mass density of an isotropic particle is defined as the mass of the particle divided by the minimum sphere envelope volume within which it can be enclosed.

Tap density can be measured by using instruments known to those skilled in the art such as the Dual Platform Microprocessor Controlled Tap Density Tester (Vankel, N.C.) or a Geopyc.TM. instrument (Micrometrics Instrument Corp., Norcross, Ga. 30093). Tap density is a standard measure of the envelope mass density. Tap density can be determined using the method of USP Bulk Density and Tapped Density, United States Pharmacopia convention, Rockville, Md., 10.sup.th Supplement, 4950-4951, 1999. Features which can contribute to low tap density include irregular surface texture and porous structure.

The diameter of the particles, for example, their VMGD, can be measured using an electrical zone sensing instrument such as a Multisizer IIe, (Coulter Electronic, Luton, Beds, England), or a laser diffraction instrument (for example Helos, manufactured by Sympatec, Princeton, N.J.). Other instruments for measuring particle diameter are well known in the art. The diameter of particles in a sample will range depending upon factors such as particle composition and methods of synthesis. The distribution of size of particles in a sample can be selected to permit optimal deposition within targeted sites within the respiratory tract.

The particles may be fabricated with the appropriate material, surface roughness, diameter and tap density for localized delivery to selected regions of the respiratory tract such as the deep lung or upper or central airways. For example, higher density or larger particles may be used for upper airway delivery, or a mixture of varying sized particles in a sample, provided with the same or different therapeutic agent may be administered to target different regions of the lung in one administration. Particles having an aerodynamic diameter ranging from about 3 to about 5 microns are preferred for delivery to the central and upper airways. Particles having an aerodynamic diameter ranging from about 1 to about 3 microns are preferred for delivery to the deep lung.

Inertial impaction and gravitational settling of aerosols are predominant deposition mechanisms in the airways and acini of the lungs during normal breathing conditions. Edwards, D. A., J. Aerosol Sci., 26: 293-317 (1995). The importance of both deposition mechanisms increases in proportion to the mass of aerosols and not to particle (or envelope) volume. Since the site of aerosol deposition in the lungs is determined by the mass of the aerosol (at least for particles of mean aerodynamic diameter greater than approximately 1 micron), diminishing the tap density by increasing particle surface irregularities and particle porosity permits the delivery of larger particle envelope volumes into the lungs, all other physical parameters being equal.

The aerodyanamic diameter can be calculated to provide for maximum deposition within the lungs, previously achieved by the use of very small particles of less than about five microns in diameter, preferably between about one and about three microns, which are then subject to phagocytosis. Selection of particles which have a larger diameter, but which are sufficiently light (hence the characterization “aerodynamically light”), results in an equivalent delivery to the lungs, but the larger size particles are not phagocytosed. Improved delivery can be obtained by using particles with a rough or uneven surface relative to those with a smooth surface.

Suitable particles can be fabricated or separated, for example by filtration or centrifugation, to provide a particle sample with a preselected size distribution. For example, greater than about 30%, 50%, 70%, or 80% of the particles in a sample can have a diameter within a selected range of at least about 5 microns. The selected range within which a certain percentage of the particles must fall may be for example, between about 5 and about 30 microns, or optimally between about 5 and about 15 microns. In one preferred embodiment, at least a portion of the particles have a diameter between about 9 and about 11 microns. Optionally, the particle sample also can be fabricated wherein at least about 90%, or optionally about 95% or about 99%, have a diameter within the selected range. The presence of the higher proportion of the aerodynamically light, larger diameter particles in the particle sample enhances the delivery of therapeutic or diagnostic agents incorporated therein to the deep lung. Large diameter particles generally mean particles having a median geometric diameter of at least about 5 microns.

The preferred particles to target antigen presenting cells (“APC”) have a minimum diameter of 400 nm, the limit for phagocytosis by APCs. The preferred particles to traffic through tissues and target cells for uptake have a minimum diameter of 10 nm. The final formulation may form a dry powder that is suitable for pulmonary delivery and stable at room temperature.

Antigenic Agents

Antigenic agents are chemical compounds, natural polymers, synthetic polymers, or biomolecules that illicit, promote, repress or otherwise stimulate immune responses in host organisms. Preferred antigenic agents for vaccines are lipids, glycolipids, polysaccharides, peptides, proteins, glycoprotein, cytokines, and/or nucleic acids. Nucleic acid antigenic agents can encode other protein antigens, enzymes that affect cellular metabolism, peptides that affect cellular communication; they can promote or interfere with cellular mechanisms, or directly stimulate a host's immune system.

The preferred malarial protein antigenic agents are the recombinant proteins CSP, AMA-1, MSP-1, and FALVAC-1. These recombinant proteins have been extensively studied for use in malaria vaccines and are known to elicit immune responses in humans.

A major difficulty in developing peptide based vaccines against malaria is the polymorphism inherent in the parasite's presentation of surface antigens. An individual parasite may express many different versions of the same surface protein concurrently or in the successive waves of blood-stage infection as a mechanism of avoiding the host immune response. Therefore, vaccines composed of multiple antigens from different stages in the life-cycle are thought to hold greater promise than single stage vaccines and provides a basic rationale underlying our invention. Alternatively, it is thought that multiple branches of the immune system will require stimulus.

Surrogate to vaccines derived from live vectors, deactivated organisms, or recombinant proteins are nucleic acid based vaccines. These “gene vaccines” involve the delivery of DNA or RNA encoding antigens into cells and make their products available to the MHC class I immune response. Nucleic acid vaccines raise the possibility of specifically stimulating the T cell response in a selective way. It has been shown that intramuscular injection of naked DNA plasmids encoding influenza antigens protect against infection from the influenza virus and specifically induce the cellular immune response (J J Donnelly, J B Ulmer, M A Liu. Ann N Y Acad Sci. 1995). Again this provides a basic rationale behind our invention.

Formulations

In the preferred embodiment, particulate malaria vaccine formulations contain mixtures of peptides, proteins, small molecules, and nucleic acid antigenic agents. Specific embodiments include aggregate nanoparticle formulations of MSP-1 alone, AMA-1 alone, MSP-1 co-formulated with MSP-1 plamid DNA, and AMA-1 co-formulated with AMA-1 plasmid DNA. These can be administered separately, in combination, or sequentially. The formulation loading is 5-50% antigen by particle weight with equal proportion protein and DNA in co-formulations. This is based upon dosage estimates required to illicit immunity. The formulated particles have a diameter of greater than 10 nm and an aggregate diameter of less than 50 um.

In the preferred embodiment, aggregate nanoparticles are in the aerodynamic range of 1-5 microns diameter and fly deep into the lungs. As the aggregate particles degrade in the body, MSP-1 and AMA-1 proteins are released into the blood stimulating a humoural immune response. The individual particles in the range of 0.1 micron are preferentially phagocytosed by APCs which express the proteins encoded by AMA-1 and MSP-1 plasmid DNA thereby initiating the cellular immune response that is necessary for a complete immunity.

III. Methods of Administration

The particles can be administered by any of several routes, including injection, oral, and topically to mucosal surfaces, but pulmonary delivery is preferred. Pulmonary administration can typically be completed without the need for medical intervention (self-administration), the pain often associated with injection therapy is avoided, and the amount of enzymatic and pH mediated degradation of the bioactive agent, frequently encountered with oral therapies, can be significantly reduced. In addition, the lungs provide a large mucosal surface for drug absorption and there is no first-pass liver effect of absorbed drugs. Further, it has been shown that high bioavailability of many molecules, for example, protein and polysaccharide macromolecules, can be achieved via pulmonary delivery or inhalation. Typically, the deep lung, or alveoli, is the primary target of inhaled bioactive agents, particularly for agents requiring systemic delivery. The lungs are also lined with phagocytic cells of the immune system and provide a means for introducing antigen to a large number of immune cells immediately following administration.

“Pulmonary delivery,” as that term is used herein refers to delivery to the respiratory tract. The “respiratory tract,” as defined herein, encompasses the upper airways, including the oropharynx and larynx, followed by the lower airways, which include the trachea followed by bifurcations into the bronchi and bronchioli (e.g., terminal and respiratory). The upper and lower airways are called the conducting airways. The terminal bronchioli then divide into respiratory bronchioli which then lead to the ultimate respiratory zone, namely, the alveoli, or deep lung. The deep lung, or alveoli, are typically the desired the target of inhaled therapeutic formulations for systemic drug delivery.

In a preferred embodiment, particles are administered via a dry powder inhaler (DPI). Metered-dose-inhalers (MDI), nebulizers or instillation techniques also can be employed. Various suitable devices and methods of inhalation which can be used to administer particles to a patient's respiratory tract are known in the art. For example, suitable inhalers are described in U.S. Pat. No. 4,069,819, issued Aug. 5, 1976 to Valentini, et al., U.S. Pat. No. 4,995,385 issued Feb. 26, 1991 to Valentini, et al., and U.S. Pat. No. 5,997,848 issued Dec. 7, 1999 to Patton, et al. Various suitable devices and methods of inhalation which can be used to administer particles to a patient's respiratory tract are known in the art. For example, suitable inhalers are described in U.S. Pat. Nos. 4,995,385, and 4,069,819 issued to Valentini, et al., U.S. Pat. No. 5,997,848 issued to Patton. Other examples include, but are not limited to, the Spinhaler.RTM. (Fisons, Loughborough, U.K.), Rotahaler.RTM. (Glaxo-Wellcome, Research Triangle Technology Park, N.C.), FlowCaps.RTM.) (Hovione, Loures, Portugal), Inhalator.RTM. (Boehringer-Ingelheim, Germany), and the Aerolizer.RTM. (Novartis, Switzerland), the diskhaler (Glaxo-Wellcome, RTP, N.C.) and others, such as known to those skilled in the art. Preferably, the particles are administered as a dry powder via a dry powder inhaler.

A receptacle encloses or stores particles/and or respirable pharmaceutical compositions comprising the particles. In one embodiment, the particles have a mass of at least 5 milligrams. In another embodiment, the mass of the particles stored or enclosed of at least about 1 mg and to at least about 100 mg. The particles can be composed of 1-100% antigenic material. In the preferred embodiment, the particles contain 5-10% antigen material by weight.

Preferably, particles administered to the respiratory tract travel through the upper airways (oropharynx and larynx), the lower airways which include the trachea followed by bifurcations into the bronchi and bronchioli and through the terminal bronchioli which in turn divide into respiratory bronchioli leading then to the ultimate respiratory zone, the alveoli or the deep lung. In a preferred embodiment of the invention, most of the mass of particles deposits in the deep lung. In another embodiment of the invention, delivery is primarily to the central airways. Delivery to the upper airways can also be obtained.

As used herein, the term “effective amount” means the amount needed to achieve the desired therapeutic or diagnostic effect or efficacy. The actual effective amounts of drug can vary according to the specific drug or combination thereof being utilized, the particular composition formulated, the mode of administration, and the age, weight, condition of the patient, and severity of the symptoms or condition being treated. Dosages for a particular patient can be determined by one of ordinary skill in the art using conventional considerations, (e.g. by means of an appropriate, conventional pharmacological protocol).

Aerosol dosage, formulations and delivery systems also may be selected for a particular therapeutic application, as described, for example, in Gonda, I. “Aerosols for delivery of therapeutic and diagnostic agents to the respiratory tract,” in Critical Reviews in Therapeutic Drug Carrier Systems, 6: 273-313, 1990; and in Moren, “Aerosol dosage forms and formulations,” in: Aerosols in Medicine. Principles, Diagnosis and Therapy, Moren, et al., Eds, Esevier, Amsterdam, 1985.

For delivery by injection, the particles are first suspended in an appropriate delivery liquid and delivered through a narrow gauge needle subcutaneously or intramuscularly to form a reservoir in vivo.

Administration of particles releasing protein and DNA replicates the immune response elicited by DNA/protein prime/boost immunizations. The DNA containing particles constitute the initial prime, and the sustained release of the protein stimulates the immune system in the same manner as the boost routines. The advantage of this technique is that is provides a single vaccination to provide long lasting immunity to the malaria or other parasite.

1. A particulate vaccine formulation comprising a mixture of peptides and/or small molecule adjuvants and/or proteins and/or nucleic acid antigenic agents.

2. The formulation of claim 1 comprising nanoparticulates.

3. The formulation of claim 2 comprising aggregates of the nanoparticulates.

4. The formulation of claim 1 wherein the protein and nucleic acid antigenic agents are released at different times from the formulation.

5. The formulation of claim 1 wherein the formulation provides sustained release of antigenic agent.

6. The formulation of claim 1 wherein the antigenic agents are or encode malarial antigenic agents.

7. A method of making a particulate vaccine formulation comprising making nanoparticles or microparticles comprising protein and nucleic acid antigens.

8. The method of claim 7 wherein the particles are made by spray drying.

9. The method of claim 7 wherein the particles comprise lipid and antigen.

10. A method of vaccination comprising administering an effective amount of a particulate vaccine formulation comprising a mixture of peptides and/or small molecule adjuvants and/or proteins and/or nucleic acid antigenic agents.

11. The method of claim 10 wherein the formulation is administered by the pulmonary route.

12. The method of claim 10 wherein the formulation is administered by injection.

13. The method of claim 10 wherein the formulation is orally administered.
